(19) United States
(12) Patent Application Publication
(10) Pub. No.: US 2022/0110856 A1
(43) Pub. Date: Apr. 14, 2022

(54) A TRANSPARENT THICKENING BLEND

(71) Applicant: Dow Global Technologies LLC, Midland, MI (US)

(72) Inventor: Daniel Almeida, Paulinia (BR)

(21) Appl. No.: 17/261,688

(22) PCT Filed: Aug. 15, 2019
(86) PCT No.: PCT/US2019/046604

§ 371 Date: Jan. 20, 2021

Related U.S. Application Data
(60) Provisional application No. 62/721,094, filed on Aug. 22, 2018.

(51) Int. Cl. A61K 8/891; A61Q 19/00; A61K 8/73; A61K 8/34

(52) U.S. Cl. CPC A61K 8/891; A61Q 19/00; A61K 2800/48; A61K 8/342; A61K 2800/10; A61K 8/731

(57) ABSTRACT

A transparent thickening blend for use in personal care formulations is provided having 1 to 10 wt %, based on weight of the transparent thickening blend, of a carbohydrate-based gelling agent; 0 to 79 wt %, based on weight of the transparent thickening blend, of a polar oil; 20 to 99 wt %, based on weight of the transparent thickening blend, of a short chain silanol having structure (I)

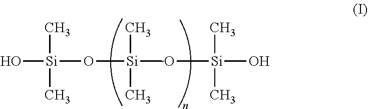

wherein n is an average of 1 to 40; and 0 to 30 wt %, based on weight of the transparent thickening blend, of an organo modified silicone.

The present invention relates to a transparent thickening blend for use in personal care formulations, comprising: 1 to 10 wt %, based on weight of the transparent thickening blend, of a carbohydrate-based gelling agent; 0 to 79 wt %, based on weight of the transparent thickening blend, of a polar oil; 20 to 99 wt %, based on weight of the transparent thickening blend, of a short chain silanol having structure (I)

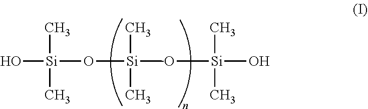

wherein n is an average of 1 to 40; and 0 to 30 wt %, based on weight of the transparent thickening blend, of an organo modified silicone.

Carbohydrate-based gelling agents (e.g., ethylcellulose) have been identified as a hydrophobic film forming agent and a water insoluble polymer component of cosmetic and pharmaceutical formulations, wherein the inclusion of the carbohydrate-based gelling agents provide enhanced adhesion, durability, hydrophobicity and viscosity to the formulations. For example, inclusion of carbohydrate-based gelling agents in personal care formulations for application to lips and skin have exhibited enhanced adhesion of such formulations and inhibit the migration of the formulation beyond the areas to which the formulations are initially applied. Such adhesion represents a desirable feature for consumers particularly for personal care formulations including lipstick, lip gloss and other lip and skin care formulations.

In addition, incorporation of carbohydrate-based gelling agents (e.g., ethylcellulose) have also been found to enhance the durability efficacy of both cosmetic formulations and pharmaceutical formulations applied to the skin or lips and therefor improve the endurance of the formulation and inhibit (a) the undesirable transfer of the formulation to items coming into contact with treated skin or lips (e.g., drinking glasses, clothing) and (b) the undesirable migration of the formulation to skin adjacent to the treated area (e.g., from the lips). Given that many personal care formulations for application to the skin or lips contain coloring agents (or other stain causing agents) the inhibition or elimination of undesirable transfers are a particularly desirable feature for consumers.

Moreover, incorporation of carbohydrate-based gelling agents (e.g., ethylcellulose) have been observed to enhance the water resistance of personal care formulations, which is a particularly desirable feature for consumers with respect to, for example, sun screen formulations.

Notwithstanding, carbohydrate-based gelling agents (e.g., ethylcellulose) have very limited solubility in most solvents utilized in personal care formulations. Typically, lower monohydric alcohols with 2 to 9 carbon atoms per molecule (e.g., ethanol, butanol, methanol, isopropanol) are employed to solubilize effective amounts of carbohydrate-based gelling agents for incorporation into personal care formulations. Such monohydric alcohol solvents have been identified as irritants and volatile. Hence the incorporation of such monohydric alcohol solvents in personal care formulations is not favored.

An approach to incorporating carbohydrate-based gelling agents into personal care formulations without using lower monohydric alcohols is disclosed in U.S. Pat. No. 5,908,631 to Arnaud et al. Arnaud et al. disclose a composition adapted for topical use on human tissue selected from the group consisting of lips and skin, said composition comprising a commercially acceptable carrier and from about 0.05 to 20% by weight ethylcellulose, said ethylcellulose being solubilized in at least one solvent, wherein the at least one solvent comprises greater than 10 weight percent of the composition, and the composition is free of monohydric alcohol.

Notwithstanding, there remains a continuing need for approaches for incorporating carbohydrate-based gelling agents into personal care formulations.

The present invention provides a transparent thickening blend for use in personal care formulations, comprising: 1 to 10 wt %, based on weight of the transparent thickening blend, of a carbohydrate-based gelling agent; 0 to 79 wt %, based on weight of the transparent thickening blend, of a polar oil; 20 to 99 wt %, based on weight of the transparent thickening blend, of a short chain silanol having structure (I)

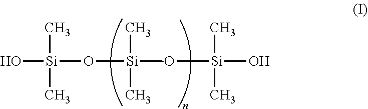

wherein n is an average of 1 to 40; and 0 to 30 wt %, based on weight of the transparent thickening blend, of an organo modified silicone.

The present invention provides a transparent thickening blend for use in personal care formulations, comprising: 1 to 10 wt %, based on weight of the transparent thickening blend, of a carbohydrate-based gelling agent; 2.5 to 75 wt %, based on weight of the transparent thickening blend, of a polar oil; 25 to 95 wt %, based on weight of the transparent thickening blend, of a short chain silanol having structure (I), wherein n is an average of 1 to 40; and 0 to 30 wt %, based on weight of the transparent thickening blend, of an organo modified silicone.

The present invention provides a transparent thickening blend for use in personal care formulations, comprising: 1 to 10 wt %, based on weight of the transparent thickening blend, of a carbohydrate-based gelling agent, wherein the gelling agent is ethylcellulose; 0 to 79 wt %, based on weight of the transparent thickening blend, of a polar oil, wherein the polar oil is octyldodecanol; 20 to 99 wt %, based on weight of the transparent thickening blend, of a short chain silanol having structure (I), wherein n is an average of 2 to 30; and 0 to 30 wt %, based on weight of the transparent thickening blend, of an organo modified silicone; wherein the transparent thickening blend has a weight basis blend ratio of the polar oil to the short chain silanol of 75:25 to 0:100; wherein the transparent thickening blend contains less than a detectable limit of an amino acid based gelatinizing agent; wherein the transparent thickening blend contains less than a detectable limit of lower aliphatic alcohols, wherein the lower aliphatic alcohols have ≤nine carbon atoms per molecule; and wherein the transparent thickening blend contains less than a detectable limit of polyhydroxystearic acid.

The present invention provides a transparent thickening blend for use in personal care formulations, comprising: 1 to 10 wt %, based on weight of the transparent thickening blend, of a carbohydrate-based gelling agent, wherein the gelling agent is ethylcellulose; 0 to 79 wt %, based on weight of the transparent thickening blend, of a polar oil, wherein the polar oil is octyldodecanol; 20 to 99 wt %, based on weight of the transparent thickening blend, of a short chain silanol having structure (I), wherein n is an average of 4 to 26; and 0 to 30 wt %, based on weight of the transparent thickening blend, of an organo modified silicone; wherein the transparent thickening blend has a weight basis blend ratio of the polar oil to the short chain silanol of 75:25 to 0:100; wherein the transparent thickening blend contains less than a detectable limit of an amino acid based gelatinizing agent; wherein the transparent thickening blend contains less than a detectable limit of lower aliphatic alcohols, wherein the lower aliphatic alcohols have ≤nine carbon atoms per molecule; and wherein the transparent thickening blend contains less than a detectable limit of polyhydroxystearic acid.

### DETAILED DESCRIPTION

We have now found that blends of polar oil with short chain silanol having structure (I), wherein n is an average of 1 to 40; wherein the blend has a weight basis blend ratio of the polar oil to the short chain silanol of 75:25 to 0:100; effectively solubilize 1 to 10 wt % of a carbohydrate-based gelling agent to form a transparent thickening blend designed for use in personal care formulations; wherein the transparent thickening blend is easily processed at atmospheric pressure (1 atm) and temperatures below 100° C.

Unless otherwise indicated, ratios, percentages, parts, and the like are by weight.

The term “cosmetically acceptable” as used herein and in the appended refers to ingredients typically used in personal care compositions, and is intended to underscore that materials that are toxic when present in the amounts typically found in personal care compositions are in hair care compositions are not contemplated as part of the present invention.

Preferably, the transparent thickening blend for use in personal care formulations of the present invention, comprises: 1 to 10 wt % (preferably, 2 to 10 wt %; more preferably, 4 to 8 wt %; still more preferably, 5 to 7 wt %; most preferably, 5.5 to 6.5 wt %), based on weight of the transparent thickening blend, of a carbohydrate-based gelling agent; 0 to 79 wt % (preferably, 2.5 to 75 wt %; more preferably, 5 to 70 wt %; still more preferably, 15 to 60 wt %; most preferably, 25 to 44 wt %), based on weight of the transparent thickening blend, of a polar oil; 20 to 99 wt % (preferably, 25 to 95 wt %; more preferably, 25 to 90 wt %; still more preferably, 25 to 85 wt %; most preferably, 25 to 80 wt %), based on weight of the transparent thickening blend, of a short chain silanol having structure (I)

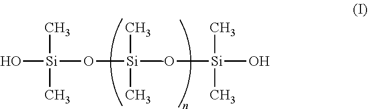

wherein n is an average of 1 to 40 (preferably, 2 to 35; more preferably, 2 to 30; most preferably, 4 to 26); and 0 to 30 wt % (preferably, 0 to 23 wt %; more preferably, 0 to 20 wt %; most preferably, 0 wt %), based on weight of the transparent thickening blend, of an organo modified silicone.

Preferably, the transparent thickening blend for use in personal care formulations of the present invention, comprises: 1 to 10 wt % (preferably, 2 to 10 wt %; more preferably, 4 to 8 wt %; still more preferably, 5 to 7 wt %; most preferably, 5.5 to 6.5 wt %), based on weight of the transparent thickening blend, of a carbohydrate-based gelling agent, wherein the carbohydrate-based gelling agent is ethylcellulose; 0 to 79 wt % (preferably, 2.5 to 75 wt %; more preferably, 5 to 70 wt %; still more preferably, 15 to 60 wt %; most preferably, 25 to 44 wt %), based on weight of the transparent thickening blend, of a polar oil, wherein the polar oil is octyldodecanol; 20 to 99 wt % (preferably, 25 to 95 wt %; more preferably, 25 to 90 wt %; still more preferably, 25 to 85 wt %; most preferably, 25 to 80 wt %), based on weight of the transparent thickening blend, of a short chain silanol having structure (I), wherein n is an average of 1 to 40 (preferably, 2 to 35; more preferably, 2 to 30; most preferably, 4 to 26); and 0 to 30 wt % (preferably, 0 to 23 wt %; more preferably, 0 to 20 wt %; most preferably, 0 wt %), based on weight of the transparent thickening blend, of an organo modified silicone; wherein the transparent thickening blend has a weight basis blend ratio of the polar oil to the short chain silanol of 75:25 to 0:100; wherein the transparent thickening blend contains less than a detectable limit of an amino acid based gelatinizing agent; wherein the transparent thickening blend contains less than a detectable limit of lower aliphatic alcohols, wherein the lower aliphatic alcohols have ≤nine carbon atoms per molecule; and wherein the transparent thickening blend contains less than a detectable limit of polyhydroxystearic acid.

Preferably, the transparent thickening blend of the present invention, comprises: 1 to 10 wt % (preferably, 2 to 10 wt %; more preferably, 4 to 8 wt %; still more preferably, 5 to 7 wt %; most preferably, 5.5 to 6.5 wt %), based on weight of the transparent thickening blend, of a carbohydrate-based gelling agent. More preferably, the transparent thickening blend of the present invention, comprises: 1 to 10 wt % (preferably, 2 to 10 wt %; more preferably, 4 to 8 wt %; still more preferably, 5 to 7 wt %; most preferably, 5.5 to 6.5 wt %), based on weight of the transparent thickening blend, of a carbohydrate-based gelling agent, wherein the carbohydrate-based gelling agent is a cellulosic polymer. Still more preferably, the transparent thickening blend of the present invention, comprises: 1 to 10 wt % (preferably, 2 to 10 wt %; more preferably, 4 to 8 wt %; still more preferably, 5 to 7 wt %; most preferably, 5.5 to 6.5 wt %), based on weight of the transparent thickening blend, of a carbohydrate-based gelling agent, wherein the carbohydrate-based gelling agent is a cellulosic polymer; wherein the cellulosic polymer is selected from the group consisting of a cellulose ether. Most preferably, the transparent thickening blend of the present invention, comprises: 1 to 10 wt % (preferably, 2 to 10 wt %; more preferably, 4 to 8 wt %; still more preferably, 5 to 7 wt %; most preferably, 5.5 to 6.5 wt %), based on weight of the transparent thickening blend, of a carbohydrate-based gelling agent, is an ethylcellulose.

Preferably, the ethylcellulose used in the transparent thickening blend of the present invention has an ethoxyl substitution of 40 to 55 percent. More preferably, the ethylcellulose used in the transparent thickening blend of the present invention has an ethoxyl substitution of 43 to 53 percent. Most preferably, the ethylcellulose used in the transparent thickening blend of the present invention has an ethoxyl substitution of 45 to 51 percent. The percent ethoxyl substitution is based on the weight of the substituted product and determined according to a Zeisel gas chromatographic technique as described in ASTM D4794-94 (2003).

Preferably, the ethylcellulose used in the transparent thickening blend of the present invention has a viscosity of 50 to 400 mPa·s (more preferably, 70 to 300 mPa·s; still more preferably, 75 to 200 mPa·s; yet still more preferably, 80 to 150 mPa·s; most preferably, 85 to 120 mPa·s) measured as a 5 weight percent solution at 25° C. in a mixture of 80 volume percent toluene and 20 volume percent ethanol. Preferably, the viscosity is measured using Ubbelohde tubes as outlined in ASTM D914-00 and as further described in ASTM D446-04, which is referenced in ASTM D914-00.

Preferably, the transparent thickening blend of the present invention, comprises: 0 to 79 wt % (preferably, 2.5 to 75 wt %; more preferably, 5 to 70 wt %; still more preferably, 15 to 60 wt %; most preferably, 25 to 44 wt %), based on weight of the transparent thickening blend, of a polar oil. More preferably, the transparent thickening blend of the present invention, comprises: 0 to 79 wt % (preferably, 2.5 to 75 wt %; more preferably, 5 to 70 wt %; still more preferably, 15 to 60 wt %; most preferably, 25 to 44 wt %), based on weight of the transparent thickening blend, of a polar oil; wherein the polar oil is a C₁₀₋₁₀₀ alkyl alcohol. Still more preferably, the transparent thickening blend of the present invention, comprises: 0 to 79 wt % (preferably, 2.5 to 75 wt %; more preferably, 5 to 70 wt %; still more preferably, 15 to 50 wt %; most preferably, 25 to 40 wt %), based on weight of the transparent thickening blend, of a polar oil, wherein the polar oil is selected from the group consisting of octyldodecanol, hexyl decanol, octyldecanol, hexyl dodecanol, oleyl alcohol, cetyl alcohol, isostearyl alcohol, undecylpentadecanol, hexylene glycol, 1,2-hexanediol, propylene glycol, glycerine, oleic alcohol, phenylethyl alcohol, ricin oil, ethyl myristate, isopropyl palmitate, finsolv, diisopropyl Sebacate, diisopropyl adipate, propylene glycol dicaprate/dicaprylate. Most preferably, the transparent thickening blend of the present invention, comprises: 0 to 79 wt % (preferably, 2.5 to 75 wt %; more preferably, 5 to 70 wt %; still more preferably, 15 to 50 wt %; most preferably, 25 to 40 wt %), based on weight of the transparent thickening blend, of a polar oil, wherein the polar oil is octyldodecanol.

Preferably, the transparent thickening blend of the present invention, comprises: 20 to 99 wt % (preferably, 25 to 95 wt %; more preferably, 25 to 90 wt %; still more preferably, 25 to 85 wt %; most preferably, 25 to 80 wt %), based on weight of the transparent thickening blend, of a short chain silanol having structure (I)

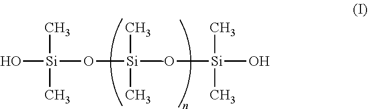

wherein n is an average of 1 to 40 (preferably, 2 to 35; more preferably, 2 to 30; most preferably, 4 to 26).

Preferably, the transparent thickening blend of the present invention, comprises: 25 to 70 wt %, based on weight of the transparent thickening blend, of a short chain silanol having structure (I), wherein n is an average of 20 to 30 (preferably, 25).

Preferably, the transparent thickening blend of the present invention, comprises: 75 to 95 wt %, based on weight of the transparent thickening blend, of a short chain silanol having structure (I), wherein n is an average of 4 to 6 (preferably, 5).

Preferably, the transparent thickening blend of the present invention has a weight basis blend ratio of the polar oil to the short chain silanol of 75:25 to 0:100. More preferably, the transparent thickening blend of the present invention has a weight basis blend ratio of the polar oil to the short chain silanol of 20:80 to 0:100. Most preferably, the transparent thickening blend of the present invention has a weight basis blend ratio of the polar oil to the short chain silanol of 20:80 to 10:90.

Preferably, the transparent thickening blend of the present invention, comprises: 0 to 30 wt % (preferably, 0 to 23 wt %; more preferably, 0 to 20 wt %; most preferably, 0 wt %), based on weight of the transparent thickening blend, of an organo modified silicone (e.g., caprylyl methicone).

Preferably, the transparent thickening blend of the present invention, contains <4 wt % (more preferably, <0.1 wt %; still more preferably, <0.05 wt %; yet still more preferably, <0.01 wt %; most preferably, less than detectible limit), based on weight of the transparent thickening blend, of an amino acid based gelatinizing agent. Examples of amino acid based gelatinizing agents include N-acetyl glutamic acid, dilauryl amide, N-caproyl glutamic acid dibutyl amide, N-lauryl glutamic acid dihexyl amide, N-lauroyl-glutamic acid dihexyl amide, N-acetyl amino acid amides, N-lauroyl-L-glutamic acid dibutyl amide (aka dibutyl lauroyl glutamide) and N-2-ethylhexanoyl-L-glutamic acid dibutyl) amide.

Preferably, the transparent thickening blend of the present invention, contains <20 wt % (preferably, <10 wt %; more preferably, <1 wt %; still more preferably, <0.1 wt %; most preferably, less than detectible limit), based on weight of the transparent thickening blend, of lower aliphatic alcohols; wherein the lower aliphatic alcohols have ≤nine carbon atoms per molecule (e.g., ethanol, propanol, isopropanol, butanol).

Preferably, the transparent thickening blend of the present invention, contains <0.1 wt % (preferably, <0.05 wt %; more preferably, <0.01 wt %; still more preferably, <0.005 wt %; most preferably, less than detectible limit), based on weight of the transparent thickening blend, of polyhydroxystearic acid.

Preferably, the transparent thickening blend of the present invention, contains <5 wt % (preferably, <1 wt %; more preferably, <0.5 wt %; still more preferably, <0.01 wt %; most preferably, less than detectible limit), based on weight of the transparent thickening blend, of water.

Preferably, the transparent thickening blend of the present invention is anhydrous.

Preferably, the transparent thickening blend of the present invention can have rheological properties ranging from pourable liquids to non-pourable gels.

Preferably, the transparent thickening blend of the present invention facilitates processing at temperatures below 100° C. (preferably, below 95° C.; most preferably, below 90° C.) to form personal care formulations comprising same.

The transparent thickening blend of the present invention can be used as a component in a variety of personal care formulations including, for example, formulations for application to the skin, lips and hair to provide enhanced adhesiveness and durability.

Some embodiments of the present invention will now be described in detail in the following Examples.

### Comparative Examples C1-C13 and Examples 1-6: Preparation Thickening Blends

In each of Comparative Examples C1-C13 and Examples 1-6 the solvent components in the amounts noted in TABLE 1 were added to a round bottom flask equipped with a mechanical stirrer and a heating mantle. The flask contents were then mixed and heated to 90° C. Then ethylcellulose (Ethocel™ STD 100 premium ethylcellulose available from The Dow Chemical Company) in the amount noted in TABLE 1 was added to the flask contents with stirring (700 to 1,000 rpm), providing the product thickening blend. The mixing time for solubilization of the ethylcellulose is noted in the TABLE 1. The turbidity of the product thickening blends were observed against a black and white display chart manufactured by Leneta Company Inc. and ranked from 0 to 10, with 0 being the most transparent. The results of the turbidity observations are reported in TABLE 1.

TABLE 1

| | | Solvent Components (wt %) | | | | Dissolution | |
|---|---|---|---|---|---|---|---|
| | Ethylcellulose¹ | | Dimethiconol | | | Time | |
| Ex. | (wt %) | Octyldodecanol | 40 cS² | 25 cS³ | FZ-3196⁴ | (in min) | Turbidity |
| C1 | 6 | 84.6 | 9.4 | 0 | 0 | 28.38 | 2 |
| C2 | 6 | 65.8 | 14.1 | 0 | 14.1 | 24.32 | 2 |
| C3 | 6 | 47.0 | 23.5 | 0 | 23.5 | 29.67 | 2 |
| C4 | 6 | 18.8 | 75.2 | 0 | 0 | 37.63 | 2 |
| C5 | 6 | 14.1 | 79.9 | 0 | 0 | 31.87 | 2 |
| C6 | 6 | 84.6 | 0 | 0 | 9.4 | 35.53 | 4 |
| C7 | 6 | 84.6 | 4.7 | 0 | 4.7 | 30.71 | 4 |
| C8 | 6 | 28.2 | 32.9 | 0 | 32.9 | 28.32 | 4 |
| C9 | 6 | 9.4 | 84.6 | 0 | 0 | 49.98 | 4 |
| C10 | 6 | 65.8 | 0 | 0 | 28.2 | 26.17 | 6 |
| C11 | 6 | 9.4 | 42.3 | 0 | 37.6 | 29.78 | 8 |
| C12 | 6 | 28.2 | 0 | 0 | 65.8 | 28.62 | Phase separation |
| C13 | 6 | 9.4 | 0 | 0 | 84.6 | — | Phase separation |
| 1 | 6 | 65.8 | 28.2 | 0 | 0 | 23.48 | 0 |
| 2 | 6 | 47.0 | 47.0 | 0 | 0 | 29.53 | 0 |
| 3 | 6 | 28.2 | 65.8 | 0 | 0 | 31.97 | 0 |
| 4 | 6 | 18.8 | 0 | 75.2 | 0 | — | 0 |
| 5 | 6 | 9.4 | 0 | 84.6 | 0 | — | 0 |
| 6 | 6 | 0 | 0 | 94.0 | 0 | — | 1 |

¹Ethocel ™ STD 100 ethylcellulose available from The Dow Chemical Company
²Dimethiconol 40 hydroxy terminated poly(dimethylsiloxane) with 40 cS available from The Dow Chemical Company
³Hydroxy terminated poly(dimethylsiloxane) with 25 cS viscosity available from Sigma-Aldrich, Inc.
⁴Caprylyl branched trisiloxane available as Dowsil ™ FZ-3196 fluid from the Dow Chemical Company

We claim:

1. A transparent thickening blend for use in personal care formulations, comprising:
1 to 10 wt % %, based on weight of the transparent thickening blend, of a carbohydrate-based gelling agent;
0 to 79 wt %, based on weight of the transparent thickening blend, of a polar oil;
20 to 99 wt %, based on weight of the transparent thickening blend, of a short chain silanol having structure (I) 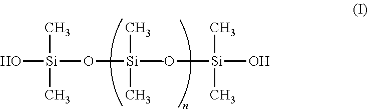
wherein n is an average of 1 to 40; and
0 to 30 wt %, based on weight of the transparent thickening blend, of an organo modified silicone.

2. The transparent thickening blend of claim 1, wherein the transparent thickening blend has a weight basis blend ratio of the polar oil to the short chain silanol of 75:25 to 0:100.

3. The transparent thickening blend of claim 1, wherein the transparent thickening blend contains less than a detectable limit of an amino acid based gelatinizing agent.

4. The transparent thickening blend of claim 1, wherein the transparent thickening blend contains less than a detectable limit of lower aliphatic alcohols, wherein the lower aliphatic alcohols have ≤nine carbon atoms per molecule.

5. The transparent thickening blend of claim 1, wherein the transparent thickening blend contains less than a detectable limit of polyhydroxystearic acid.

6. The transparent thickening blend of claim 1, wherein n is an average of 2 to 30.

7. The transparent thickening blend of claim 1, wherein the carbohydrate-based gelling agent is ethylcellulose.

8. The transparent thickening blend of claim 1, wherein the polar oil is octyldodecanol.

9. The transparent thickening blend of claim 1, wherein the carbohydrate-based gelling agent is ethylcellulose; wherein the polar oil is octyldodecanol; wherein n is an average of 2 to 35; wherein the transparent thickening blend has a weight basis blend ratio of the polar oil to the short chain silanol of 75:25 to 0:100; wherein the transparent thickening blend contains less than a detectable limit of an amino acid based gelatinizing agent; wherein the transparent thickening blend contains less than a detectable limit of lower aliphatic alcohols, wherein the lower aliphatic alcohols have ≤nine carbon atoms per molecule; and wherein the transparent thickening blend contains less than a detectable limit of polyhydroxystearic acid.

10. The transparent thickening blend of claim 9, wherein n is an average of 4 to 26.
